(19) United States
(12) Plant Patent Application Publication
(10) Pub. No.: US 2003/0079261 P1
(43) Pub. Date: Apr. 24, 2003

(54) Hybrid tea rose plant named 'Meiruchka'

(76) Inventor: Alain A. Meilland, Antibes (FR)

Correspondence Address: BURNS, DOANE, SWECKER & MATHIS, L.L.P., P.O. Box 1404, Alexandria, VA, 22313-1404, US

(21) Appl. No.: 10/306,208

(22) Filed: Nov. 29, 2002

Related U.S. Application Data
(63) Continuation of application No. 09/276,743, filed on Mar. 26, 1999, now abandoned.

(51) Int. Cl. A01H 5/00

(52) U.S. Cl. PLT/132

(57) ABSTRACT

A new and distinct variety of Hybrid Tea rose plant is provided which forms in abundance attractive brilliant bicolored double blossoms that are orange-red on the upper surface and canary yellow on the under surface and are borne on straight stems. Such blossoms possess no fragrance. The plant exhibits an erect growth habit, forms attractive dark green foliage, and exhibits good disease resistance. The new variety is particularly well suited for producing cut flowers under greenhouse growing conditions.

### SUMMARY OF THE INVENTION

The new variety of Hybrid Tea rose plant was created by artificial pollination wherein two parents were crossed which previously had been studied in the hope that they would contribute the desired characteristics. The female parent (i.e., the seed parent) of the new variety was the ‘Keiriblem’ variety (non-patented in the United States). The male parent (i.e., the pollen parent) was the ‘Krimony’ variety (U.S. Plant Pat. No. 8,845). The parentage of the new variety can be summarized as follows:

‘Keiriblem’×‘Krimony’.

The seeds resulting from the above pollination were sown and small plants were obtained which were physically and biologically different from each other. Selective study resulted in the identification of a single plant of the new variety.

It was found that the new variety of Hybrid Tea rose plant of the present invention possesses the following combination of characteristics:

(a) forms attractive dark green foliage,

(b) forms in abundance attractive brilliant bicolored double blossoms that are orange-red on the upper surface and canary yellow on the under surface and are borne on straight stems,

(c) exhibits an erect growth habit, and

(d) is particularly well suited for the production of cut flowers under greenhouse growing conditions.

The brilliant bicolored blossoms contrast nicely with the dark green foliage.

The new variety well meets the needs of the horticultural industry and exhibits good productivity during cut flower production in a greenhouse.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meiruchka’ variety. It is contemplated that this variety will be marketed in the United States under the BOLCHOI trademark.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during April while budded on Rosa indica understock and growing in greenhouses at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view obverse;

FIG. 7—illustrates a specimen of an open flower—plan view reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface; and

FIG. 16—illustrates a specimen of a leaf with seven leaflets—plan view—upper surface.

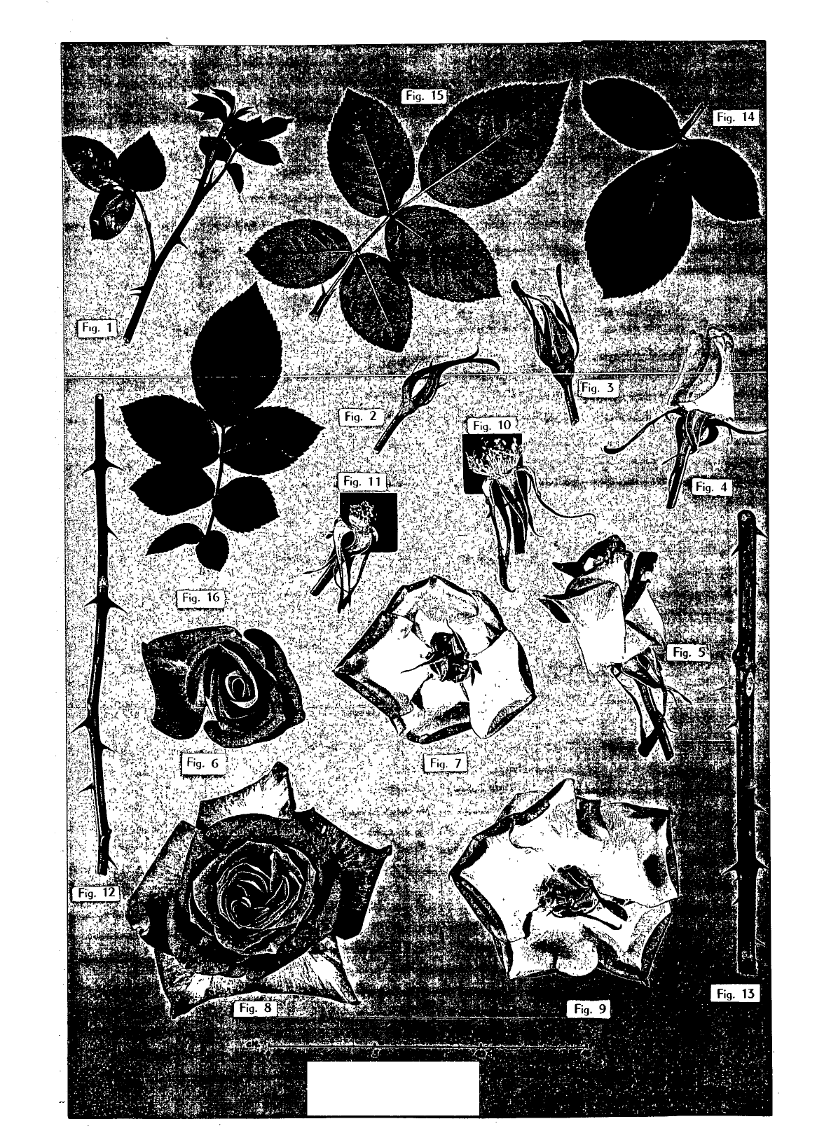

### DETAILED DESCRIPTION

The chart used in the identification of the colors is that of The Royal Horticultural Society (R.H.S. Colour Chart). The description is based on the observation of two year-old plants during April while budded on Rosa indica understock and growing indoors at Le Cannet des Maures, Var, France.

Class: Hybrid Tea.

Plant:

height.—when pruned to a height of 85 cm., floral stems of approximately 70 to 90 cm. in length commonly are produced. p2 habit.—erect.

Branches:

color.—young stems: near Green Group 137C and very lightly suffused with reddish brown. adult wood: near Green Group 137C.

thorns.—size: medium (as illustrated). quantity: fairly numerous (as illustrated). color: pinkish on young stems and pinkishgreen on adult stems. configuration: slightly curved toward the base on the upper surface, and concave with a narrow base on the under surface.

Leaves:

stipules.—adnate, pectinate, and rather broad.

petioles.—upper surface: rather glandular in nature with prickles. under surface: numerous prickles commonly are present.

leaflets.—number: 3, 5, and 7 (most often). shape: with rounded base. serration: regular (as illustrated). texture: consistent. general appearance: dense, and medium green. color (young foliage): upper surface: Yellow-Green Group 147A. under surface: Greyed-Green Group 191A. color (adult foliage): upper surface: Yellow-Green Group 147A. under surface: Greyed-Green Group 191A.

Inflorescence

number of flowers.—usually one flower per stem.

peduncle.—medium green, and the length is approximately 11.5 cm. on average.

sepals.—upper surface: greenish in coloration. under surface: non-glandular and commonly with extensions (as illustrated).

buds.—shape: conical. size: small. length: approximately 3.5 cm. on average. color upon opening: upper surface: Red Group 45A at the margins, and Yellow-Orange Group 14A at the base with an intermediate blended zone. under surface: Yellow-Orange Group 17C and suffused at the margin with Red Group 44A.

flower.—shape: with a high center. diameter: commonly approximately 11.5 cm. on average. color (when opening begins): upper surface: Red Group 44A at the margins, and Yellow-Orange Group 14A at the base with an intermediate blended area. under surface: Yellow-Orange Group 15B and lightly suffused on the margin with Red Group 43B. color (when blooming): upper surface: Yellow-Orange Group 14B towards the base, and lightly streaked with Red Group 44A towards the margin. surface: Yellow-Orange Group 14A and lightly suffused with Red Group 47B at the margin. color (at end of opening): upper surface: Yellow-Orange Group 14B at the base, yellow with some whitening and lightly streaked with Red Group 44B toward the center, and Yellow-Orange Group 14B streaked and suffused with Red Group 44B at the margin. under surface: Yellow Group 13C. fragrance: none. lasting quality: approximately 8 to 10 days on the plant and approximately 6 to 8 days when cut and placed in a vase. petal shape: rounded. petal drop: good, the petals commonly detach cleanly. stamen number: approximately 100 on average. anthers: orange-brown in coloration. filaments: orange in coloration. pistils: approximately 98 on average. stigmas: Havana brown in coloration. styles: dark pink in coloration. receptacle: in longitudinal section in the shape of a jug.

Development

vegetation.—very strong.

blooming.—abundant.

aptitude to bear fruit.—very good.

resistance to diseases.—very good.

I claim:

1. A new and distinct variety of Hybrid Tea rose plant characterized by the following combination of characteristics:
(a) forms attractive dark green foliage,
(b) forms in abundance attractive brilliant bicolored double blossoms that are orange-red on the upper surface and canary yellow on the under surface and are borne on straight stems,
(c) exhibits an erect growth habit, and
(d) is particularly well suited for the production of cut flowers under greenhouse growing conditions; substantially as herein shown and described, and the parts thereof.
